(19) United States
(12) Patent Application Publication
(10) Pub. No.: US 2014/0121194 A1
(43) Pub. Date: May 1, 2014

(54) METHOD FOR ADMINISTERING AGENT FOR CONTROLLING ECTOPARASITE TO DOG

(71) Applicant: SUMITOMO CHEMICAL COMPANY, LIMITED, Tokyo (JP)

(72) Inventors: Kaori IKARI, Takarazuka-shi (JP); Naonobu NISHIGUCHI, Takarazuka-shi (JP)

(73) Assignee: SUMITOMO CHEMICAL COMPANY, LIMITED, Tokyo (JP)

(21) Appl. No.: 14/056,074

(22) Filed: Oct. 17, 2013

(30) Foreign Application Priority Data
Nov. 1, 2012 (JP) 2012-241601

(51) Int. Cl. C07D 471/04; C07D 413/12; C07D 413/10; C07D 261/04

(52) U.S. Cl. CPC C07D 471/04; C07D 261/04; C07D 413/12; C07D 413/10
USPC 514/210.18; 514/378; 514/340; 514/256; 514/255.05; 514/300

(57) ABSTRACT

There is provided a method for controlling an animal ectoparasite having excellent controlling effects. The method for controlling an ectoparasite of dogs comprises orally administering an ectoparasite-controlling agent comprising as an active ingredient an isoxazoline compound represented by formula (I):

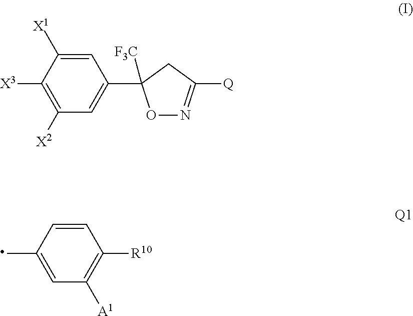

wherein X¹ represents a halogen atom or a C1-C3 haloalkyl, X² represents a hydrogen atom, a halogen atom or a C1-C3 haloalkyl, X³ represents a hydrogen atom or a halogen atom, Q represents, for example, Q1, A¹ represents R¹¹—C(═O)—N(R¹²)—N(R¹³)—, R¹¹—C(═O)—N(R¹²)—CH₂— or R¹¹—C(═O)—N(R¹²)—, and R¹⁰ represents a hydrogen atom, a halogen atom or a C1-C3 alkyl group,

to a dog in a period from 30 minutes immediately before the start of feeding to 120 minutes after the end of feeding.

### BACKGROUND OF THE INVENTION

1. Field of the Invention

The present invention relates to a method for administering an agent for controlling an ectoparasite to a dog.

2. Description of the Related Art

Heretofore, various compounds for controlling parasites living on the body surface or hair of dogs or in the vicinity thereof (so-called, ectoparasites) have been found, and methods comprising applying agents containing said compounds to the body surface of animals or orally administering the agents to animals have been known (see, for example, Patent Literature 1). However, conventional administration methods are not always sufficiently effective, and thus there is still a demand for a controlling agent and controlling method having excellent controlling effects on ectoparasites.

### PRIOR ART LITERATURE

### Patent Literature

Patent Literature 1: JP-A-2003-313104

### SUMMARY OF THE INVENTION

An object of the present invention is to provide a method for administering a certain type of an ectoparasite-controlling agent to a dog having an excellent controlling effect.

The present inventors have intensively studied for achieving the above object, and consequently found that an agent containing an isoxazoline compound represented by the following formula (I) as an active ingredient is orally administered to a dog in a predetermined time before and after feeding time of the dog, thereby showing excellent controlling effects on ectoparasites. The present invention has been accomplished thereby.

More specifically, the present invention includes the following.

[1] A method for controlling an ectoparasite of dogs, which comprises orally administering an ectoparasite-controlling agent comprising as an active ingredient an isoxazoline compound represented by formula (I):

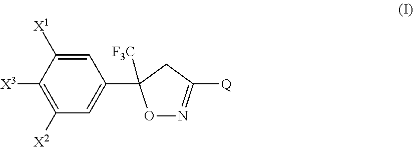

wherein X¹ represents a halogen atom or a C1-C3 haloalkyl, X² represents a hydrogen atom, a halogen atom or a C1-C3 haloalkyl, X³ represents a hydrogen atom or a halogen atom, and Q means any group selected from Q1 to Q6 as follows:

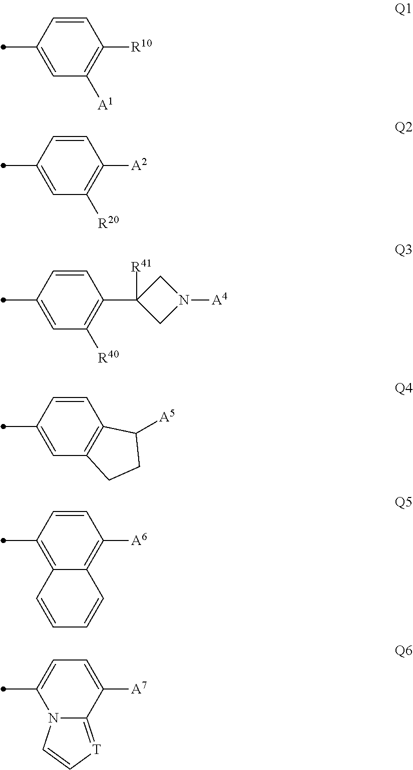

wherein

A¹ represents R¹¹—C(═O)—N(R¹²)—N(R¹³)—, R¹¹—C(═O)—N(R¹²)—CH₂— or R¹¹—C(═O)—N(R¹²)—,
R¹⁰ represents a hydrogen atom, a halogen atom or a C1-C3 alkyl,
R¹¹ represents a C1-C6 alkyl group, a C1-C6 haloalkyl group, a C3-C6 cycloalkyl group or a (C1-C6 alkoxy)C1-C6 alkyl group,
R¹² represents a hydrogen atom or a methyl group, and
R¹³ represents a hydrogen atom or a methyl group;
A² represents R²¹—N(R²²)—C(═O)—,

### R²³—N(R²⁴)—C(═O)—CH(R²⁵)—N(R²²)—C(═O)—,

### R²⁶—N(R²⁷)—N(R²²)—C(═O)—, R²⁸—N═CH—N(R²²)—C(═O)—,

### R²⁹—C(═O)—N(R³⁰)—CH(R³¹)—, R³²—O—N═C(R³³)—,

R³⁴—NH—C(═O)—CH₂O—N═C(R³³)—, R³⁴—NH—C(═O)—NH—N═C(R³³)— or R³⁵—NH—C(═NH)—NH—N═C(R³³)—,
R²⁰ represents a hydrogen atom, a halogen atom, a nitro group, an amino group, an acetylamino group or a C1-C3 alkyl group,
R²¹ represents a C1-C6 alkyl group, a C1-C6 haloalkyl group, a (hydroxy)C1-C6 alkyl group, a (C1-C6 alkoxy)C1-C6 alkyl group, a (C1-C6 alkylthio)C1-C6 alkyl group or any one heterocyclic group selected from the following group:

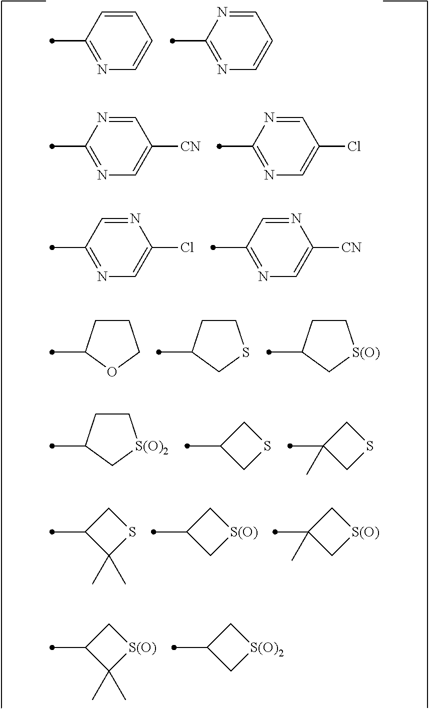

R²² represents a hydrogen atom, a (C1-C6 alkyl)carbonyl group or a (C1-C6 alkoxy)carbonyl group,
R²³ represents a C1-C6 alkyl group, a C1-C6 haloalkyl group or a (C1-C6 alkoxy)C1-C6 alkyl group,
R²⁴ represents a hydrogen atom or a C1-C3 alkyl group,
R²⁵ represents a hydrogen atom or a C1-C3 alkyl group,
R²⁶ represents a phenyl group,
R²⁷ represents a hydrogen atom or a C1-C3 alkyl group,
R²⁸ represents a C1-C3 alkoxy group,
R²⁹ represents a C1-C6 alkyl group, a C1-C6 haloalkyl group, a C3-C6 cycloalkyl group, a (C3-C6 cycloalkyl)C1-C6 alkyl group, a (C1-C6 alkoxy)C1-C6 alkyl group or a C1-C6 alkoxy group,
R³⁰ represents a hydrogen atom or a C1-C3 alkyl group,
R³¹ represents a hydrogen atom or a C1-C3 alkyl group,
R³² represents a hydrogen atom, a C1-C6 alkyl group or a C1-C6 haloalkyl group,
R³³ represents a hydrogen atom, a cyano group, a C1-C3 alkyl group, a C1-C3 alkoxy group or a (C1-C3 alkyl)carbonyl group,
R³⁴ represents a C1-C6 alkyl group, a C1-C6 haloalkyl group or a (C3-C6 cycloalkyl)C1-C6 alkyl group, and
R³⁵ represents a hydrogen atom, a C1-C6 alkyl group or a C1-C6 haloalkyl group;
A⁴ represents R⁴²—C(═O)— or R⁴²—NH—C(═O)—,
R⁴⁰ represents a hydrogen atom, a halogen atom, a nitro group, an amino group, an acetylamino group or a C1-C3 alkyl group,
R⁴¹ represents a hydrogen atom, a fluorine atom or a hydroxyl group, and
R⁴² represents a C1-C6 alkyl group, a C1-C6 haloalkyl group, a cyano(C1-C3 alkyl) group, a C3-C6 cycloalkyl group, a (C1-C6 alkoxy)C1-C6 alkyl group or a (C1-C6 alkylthio)C1-C6 alkyl group;
A⁵ represents R⁵¹—N(R⁵²)—, R⁵³—C(═O)—N(R⁵²)—,

### R⁵¹—N(R⁵²)—C(═O)—N(R⁵²)—, R⁵¹—O—C(═O)—N(R⁵²)— or R⁵³—C(═O)—,

R⁵¹ represents a C1-C6 alkyl group, a C1-C6 haloalkyl group or a C3-C6 cycloalkyl group,
R⁵² represents a hydrogen atom or a C1-C3 alkyl group,
R⁵³ represents a C1-C6 alkyl group, a C1-C6 haloalkyl group, a C3-C6 cycloalkyl group, a (hydroxy)C1-C6 alkyl group or a (C1-C6 alkoxy)C1-C6 alkyl group;
A⁶ represents R⁶¹—N(R⁶²)—C(═O)— or

### R⁶³—N(R⁶⁴)—C(═O)—CH(R⁶⁵)—N(R⁶²)—C(═O)—,

R⁶¹ represents a C1-C6 alkyl group, a C1-C6 haloalkyl group, a (hydroxy)C1-C6 alkyl group, a (C1-C6 alkoxy)C1-C6 alkyl group or a (C1-C6 alkylthio)C1-C6 alkyl group,
R⁶² represents a hydrogen atom, a (C1-C6 alkyl)carbonyl group or a (C1-C6 alkoxy)carbonyl group,
R⁶³ represents a C1-C6 alkyl group, a C1-C6 haloalkyl group or a (C1-C6 alkoxy)C1-C6 alkyl group,
R⁶⁴ represents a hydrogen atom or a C1-C6 alkyl group, and
R⁶⁵ represents a hydrogen atom or a C1-C6 alkyl group;
A⁷ represents R⁷¹—N(R⁷²)—C(═O)— or

### R⁷³—N(R⁷⁴)—C(═O)—CH(R⁷⁵)—N(R⁷²)—C(═O)—,

T represents a nitrogen atom or CR⁷⁶,
R⁷¹ represents a C1-C6 alkyl group, a C1-C6 haloalkyl group, a (hydroxy)C1-C6 alkyl group, a (C1-C6 alkoxy)C1-C6 alkyl group or a (C1-C6 alkylthio)C1-C6 alkyl group,
R⁷² represents a hydrogen atom, a (C1-C6 alkyl)carbonyl group or a (C1-C6 alkoxy)carbonyl group,
R⁷³ represents a C1-C6 alkyl group, a C1-C6 haloalkyl group or a (C1-C6 alkoxy)C1-C6 alkyl group,
R⁷⁴ represents a hydrogen atom or a C1-C6 alkyl group,
R⁷⁵ represents a hydrogen atom or a C1-C6 alkyl group, and
R⁷⁶ represents a hydrogen atom or a C1-C3 alkyl group, to a dog in a period from 30 minutes immediately before the start of feeding to 120 minutes after the end of feeding of a dog.

[2] The control method according to [1], wherein the dosage form of the ectoparasite-controlling agent is a liquid formulation, a capsule formulation, a dust, a powder, a tablet or a chewable tablet.

[3] The control method according to [1] or [2], wherein the ectoparasite is a flea, a louse or a tick.

According to the controlling method of the present invention, an ectoparasite of dogs can be effectively controlled.

### DETAILED DESCRIPTION OF THE PREFERRED EMBODIMENTS

The controlling method of the present invention is a method for controlling an ectoparasite (described as the controlling method of the present invention), which comprises orally administering an ectoparasite-controlling agent containing as an active ingredient an isoxazoline compound represented by the formula (I) (hereinafter, described as the isoxazoline compound) (hereinafter, described as the present controlling agent) in a specific administration timing i.e., in a period from 30 minutes immediately before the start of feeding and to 120 minutes after the end of feeding to a dog.

Herein,

the “halogen atom” refers to a fluorine atom, a chlorine atom, a bromine atom and an iodine atom.

The “C1-C3 alkyl group” refers to a methyl group, an ethyl group, a propyl group and an isopropyl group.

Examples of the “C1-C6 alkyl group” include a methyl group, an ethyl group, a propyl group, an isopropyl group, a butyl group, an isobutyl group, a sec-butyl group, a tert-butyl group, a pentyl group, a 1-methylbutyl group, a 2-methylbutyl group, a 3-methylbutyl group, a 1-ethylpropyl group, a 1,1-dimethylpropyl group, a 1,2-dimethylpropyl group, a 2,2-dimethylpropyl group, a hexyl group, a 1-methylpentyl group, a 2-methylpentyl group, a 1,1-dimethylbutyl group and a 1,3-dimethylbutyl group.

Examples of the “C1-C6 haloalkyl group” include a fluoromethyl group, a difluoromethyl group, a dichloromethyl group, a trifluoromethyl group, a chlorofluoromethyl group, a bromofluoromethyl group, a chlorodifluoromethyl group, a bromodifluoromethyl group, a 1-fluoroethyl group, a 1,1-difluoroethyl group, a 2,2,2-trifluoroethyl group, a 1,1,2,2,2-pentafluoroethyl group, a 3,3,3-trifluoropropyl group, a 1,1,2,2,3,3,3-heptafluoropropyl group, a 4,4,4-trifluorobutyl group and a 1,2,2,2-tetrafluoro-1-(trifluoromethyl)ethyl group.

Examples of the “C3-C6 cycloalkyl group” include a cyclopropyl group, a 1-methylcyclopropyl group, a 2-methylcyclopropyl group, a 2,2-dimethylcyclopropyl group, a cyclobutyl group, a cyclopentyl group, a 1-methylcyclopentyl group, a 2-methylcyclopentyl group, a 3-methylcyclopentyl group and a cyclohexyl group.

The “C1-C3 alkoxy group” refers to a methoxy group, an ethoxy group, a propoxy group and an isopropoxy group.

Examples of the “(hydroxy)C1-C6 alkyl group” include a 2-hydroxyethyl group, a 3-hydroxypropyl group and a 6-hydroxyhexyl group, and

examples of the “(C1-C6 alkoxy)C1-C6 alkyl group” include a methoxymethyl group, a 2-methoxyethyl group, a 3-methoxypropyl group, an ethoxymethyl group, a propoxymethyl group, a hexyloxymethyl group, a 6-methoxyhexyl group and a 1-methoxypropyl group.

Examples of the “(C1-C6 alkylthio)C1-C6 alkyl group” include a methylthiomethyl group, a 2-methylthioethyl group, a 3-methylthiopropyl group, an ethylthiomethyl group, a propylthiomethyl group, a hexylthiomethyl group, a 6-methylthiohexyl group and a 1-methylthiopropyl group.

Examples of the “(C1-C6 alkyl)carbonyl group” include a methylcarbonyl group, an ethylcarbonyl group, a propylcarbonyl group, an isopropylcarbonyl group and a hexylcarbonyl group.

Examples of the “(C1-C6 alkoxy)carbonyl group” include a methoxycarbonyl group, an ethoxycarbonyl group, a propoxycarbonyl group, an isopropoxycarbonyl group and a hexyloxycarbonyl group.

Examples of the group represented by the following formula

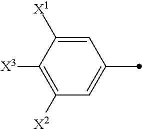

include a 3,5-dichlorophenyl group, a
3-(trifluoromethyl)phenyl group, a
4-fluoro-3,5-dichlorophenyl group, a
5-chloro-3-(trifluoromethyl)phenyl group and a
4-chloro-3-(trifluoromethyl)phenyl group.

Examples of the present isoxazoline compound are known compounds described in WO 2010/090344 A, WO 2005/085216 A, WO 2009/002809 A, WO 2009/080250 A, WO 2010/072781 A, WO 2007/105814 A, WO 2011/075591 A and WO 2012/017359 A, and can be produced by the production methods described in these publications.

Specific examples of the present isoxazoline compound include the following compounds.

The compounds represented by formula (I-1)

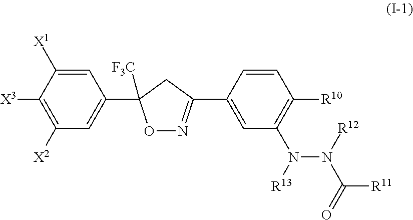

TABLE 1

| X¹ | X² | X³ | R¹⁰ | R¹³ | R¹² | R¹¹ |
|---|---|---|---|---|---|---|
| Cl | Cl | H | H | H | H | Me |
| Cl | Cl | H | H | H | Me | Me |
| Cl | Cl | H | H | Me | H | Me |
| Cl | Cl | H | H | Me | Me | Me |
| Cl | Cl | H | Cl | H | H | CH₂CHMe₂ |
| Cl | Cl | H | Cl | H | H | Me |
| Cl | Cl | H | Cl | H | H | Et |
| Cl | Cl | H | Cl | H | H | CMe₃ |
| Cl | Cl | H | Cl | Me | H | Me |
| Cl | Cl | H | Cl | H | H | Pr |
| Cl | Cl | H | Cl | H | H | Bu |
| Cl | Cl | H | Cl | H | H | Pen |
| Cl | Cl | H | H | H | H | CH₂CF₃ |
| Cl | Cl | H | Me | H | H | CH₂CF₃ |
| Cl | Cl | H | F | H | H | CH₂CF₃ |
| Cl | Cl | H | Cl | H | H | CH₂CF₃ |
| Cl | Cl | H | Cl | H | H | CH₂CH₂CF₃ |
| Cl | Cl | H | Cl | H | H | CF₃ |
| Cl | Cl | H | Cl | Me | H | CH₂CF₃ |
| Cl | Cl | H | Cl | Me | H | CH₂CH₂CF₃ |
| Cl | Cl | H | Cl | H | H | CH₂CH₂CH₂Br |
| Cl | Cl | H | Cl | H | H | CH₂CH₂CH₂CF₃ |
| Cl | Cl | H | Cl | H | H | CMe₂CH₂Cl |
| Cl | Cl | H | Cl | H | H | CH₂Br |
| Cl | Cl | H | Et | H | H | CH₂CF₃ |
| Cl | Cl | H | Cl | H | H | Pr^{c} |
| Cl | Cl | H | Cl | Me | H | Pr^{c} |
| Cl | Cl | H | Cl | H | H | CH₂OMe |

(In Tables 1 to 10, Me, Et, Pr, Pr^{c}, Bu, Bu^{c} and Pen respectively mean a methyl group, an ethyl group, a propyl group, a cyclopropyl group, a butyl group, a cyclobutyl group and a pentyl group.)

The compounds represented by formula (T-2)

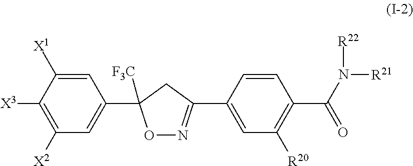

TABLE 2

| X¹ | X² | X³ | R²⁰ | R²² | R²¹ |
|---|---|---|---|---|---|
| Cl | Cl | H | Cl | H | Z16 |
| Cl | Cl | H | Br | H | Z16 |
| Cl | Cl | H | Me | H | Z16 |
| Cl | Cl | H | Me | H | Z17 |
| Cl | Cl | H | Me | H | Z18 |
| Cl | Cl | H | Me | H | Z19 |
| Cl | Cl | H | Br | H | Z13 |
| Cl | Cl | H | Me | C(═O)Me | Z13 |
| Cl | Cl | H | Me | C(═O)OMe | Z13 |
| Cl | Cl | H | Me | H | Z12 |
| Cl | Cl | H | Me | C(═O)CHMe₂ | Z12 |
| Cl | Cl | H | Me | H | Z15 |
| Cl | Cl | Cl | H | H | Z16 |
| CF₃ | Cl | Cl | H | H | Z16 |
| Cl | Cl | H | Me | H | Z21 |
| Cl | Cl | H | Me | H | Z22 |
| Cl | Cl | H | Me | H | Z20 |
| Cl | Cl | H | Me | H | Z23 |
| Cl | Cl | H | Me | H | Z24 |
| Cl | Cl | H | Me | H | Z25 |
| Cl | Cl | H | Me | H | Z26 |
| Cl | Cl | H | CF₃ | H | Z20 |
| CF₃ | Cl | H | Me | H | Z20 |
| CF₃ | CF₃ | H | Me | H | Z21 |
| CF₃ | CF₃ | H | Me | H | Z20 |
| CF₃ | CF₃ | H | Me | H | Z23 |
| Cl | Cl | H | Me | C(═O)Me | CH₂OEt |
| Cl | Cl | H | Cl | H | CH₂OCH₂CF₃ |
| Cl | Cl | H | Br | H | CH₂OCH₂CF₃ |
| Cl | Cl | H | Me | H | CH₂OCH₂CF₃ |
| Cl | Cl | H | Me | C(═O)Me | CH₂OCH₂CF₃ |

(In Table 2, Z10 to Z26 mean the following groups.)

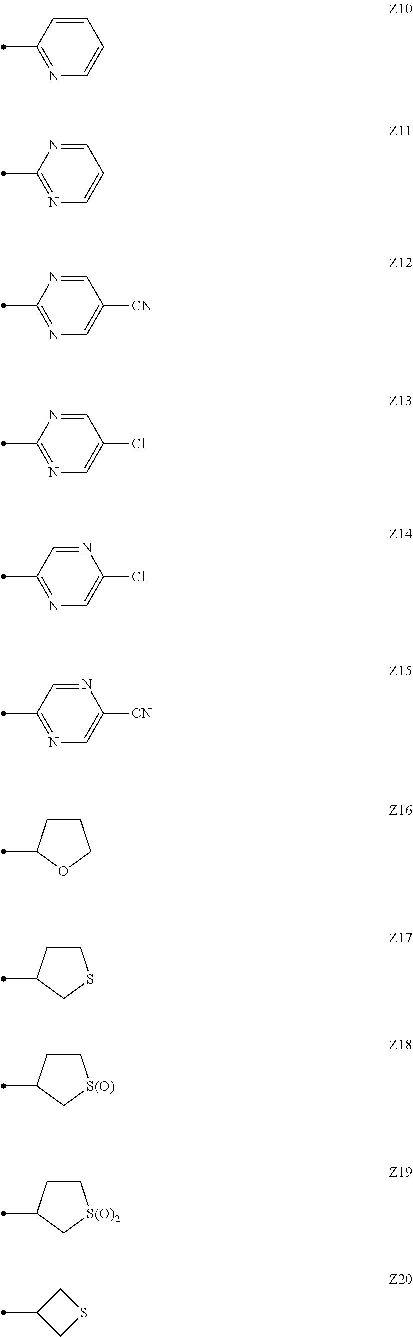

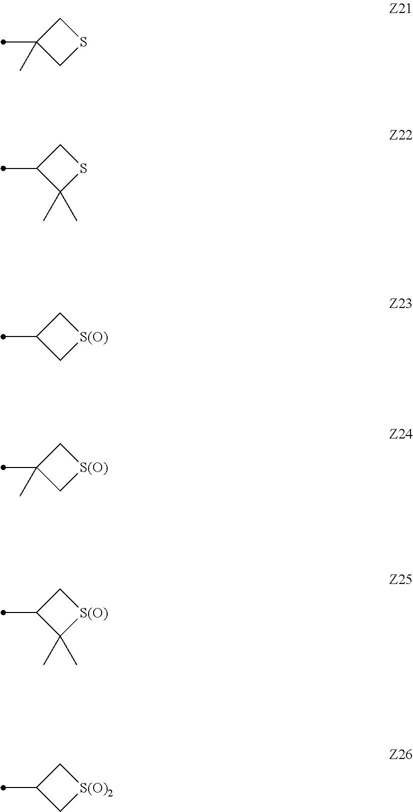

The compounds represented by formula (I-3)

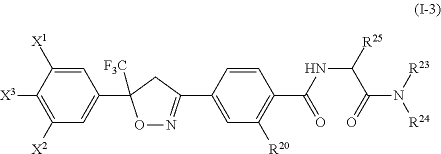

TABLE 3

| X¹ | X² | X³ | R²⁰ | R²⁵ | R²⁴ | R²³ |
|---|---|---|---|---|---|---|
| Cl | Cl | H | Cl | H | H | Et |
| Cl | Cl | H | Br | H | H | Et |
| Cl | Cl | H | I | H | H | Et |
| Cl | Cl | H | Cl | H | H | CHMe₂ |
| Cl | Cl | H | I | H | H | CHMe₂ |
| Cl | Cl | H | Cl | H | H | CH₂CH₂Cl |
| Cl | Cl | H | Br | H | H | CH₂CH₂Cl |
| Cl | Cl | H | Me | H | H | CH₂CF₃ |
| Cl | Cl | H | NO₂ | H | H | CH₂CF₃ |
| Cl | Cl | H | NH₂ | H | H | CH₂CF₃ |
| Cl | Cl | H | Cl | Me | H | Et |
| Cl | Cl | H | I | Me | H | Et |
| Cl | Cl | Cl | Cl | Me | H | CH₂CH₂Cl |
| Cl | Cl | Cl | Br | Me | H | CH₂CH₂Cl |
| Cl | Cl | H | I | Me | H | CH₂CH₂Cl |
| Cl | Cl | H | Cl | Me | H | CH₂CF₃ |
| Cl | Cl | H | Br | Me | H | CH₂CF₃ |
| Cl | Cl | H | I | Me | H | CH₂CF₃ |
| Cl | Cl | H | Me | Me | H | CH₂CF₃ |
| Cl | Cl | Cl | H | H | H | CH₂CF₃ |
| Cl | Cl | Cl | H | Me | H | CH₂CF₃ |
| CF₃ | Cl | Cl | H | H | H | CH₂CF₃ |
| CF₃ | Cl | F | Me | H | H | CH₂CF₃ |
| CF₃ | CF₃ | H | H | Me | H | CH₂CF₃ |

The compounds represented by formula (I-4)

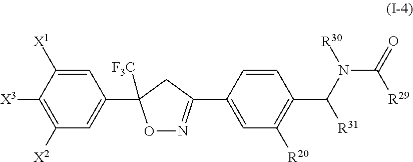

TABLE 4

| X¹ | X² | X³ | R²⁰ | R³¹ | R³⁰ | R²⁹ |
|---|---|---|---|---|---|---|
| Cl | Cl | H | H | H | H | CHMe₂ |
| Cl | Cl | H | H | H | H | Pr^{c} |
| Cl | Cl | H | H | H | H | CH₂Pr^{c} |
| Cl | Cl | H | H | H | H | CH₂Cl |
| Cl | Cl | H | H | H | H | CHF₂ |
| Cl | Cl | H | H | H | H | CH₂OMe |
| Cl | Cl | H | H | H | H | CH₂OEt |
| Cl | Cl | H | H | H | H | OEt |
| Cl | Cl | H | H | H | Me | Et |
| Cl | Cl | H | H | H | Me | Pr |
| Cl | Cl | H | H | H | Me | Pr^{c} |
| Cl | Cl | H | H | H | Me | CHMe₂ |
| Cl | Cl | H | H | H | Et | Pr |
| Cl | Cl | H | H | H | Et | Pr^{c} |
| Cl | Cl | H | H | Me | H | Pr |
| Cl | Cl | H | Cl | H | H | Et |
| Cl | Cl | H | Cl | H | H | Pr |
| Cl | Cl | H | Cl | H | H | CH₂CHMe₂ |
| Cl | Cl | H | Cl | H | H | Pr^{c} |
| Cl | Cl | H | Cl | H | H | Bu^{c} |
| Cl | Cl | H | Cl | H | H | CF₃ |
| Cl | Cl | H | Cl | H | H | CH₂CHCl |
| Cl | Cl | H | Cl | H | H | CF₂CHF₂ |
| Cl | Cl | H | Cl | H | H | CF₂CF₃ |
| Cl | Cl | H | I | H | H | Pr |
| Cl | Cl | H | I | H | H | Prc |
| Cl | Cl | H | I | H | H | CF₃ |
| Cl | Cl | H | I | H | H | CF₂CF₃ |
| Cl | Cl | H | I | H | H | OEt |
| Cl | Cl | H | Me | H | H | CHMe₂ |

The compounds represented by formula (I-5)

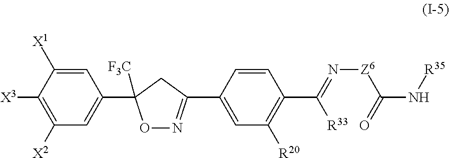

TABLE 5

| X¹ | X² | X³ | R²⁰ | R³³ | Z⁶ | R³⁵ |
|---|---|---|---|---|---|---|
| Cl | Cl | H | Me | H | OCH₂ | Et |
| Cl | Cl | H | Me | H | OCH₂ | CHMe₂ |
| Cl | Cl | H | Me | H | OCH₂ | CH₂CF₃ |
| Cl | Cl | H | Me | H | OCH₂ | CH₂Pr^{c} |
| Cl | Cl | H | Me | H | OCH₂ | Me |
| Cl | Cl | H | Me | Me | OCH₂ | Et |
| Cl | Cl | H | Me | Me | OCH₂ | CH₂CF₃ |
| Cl | Cl | H | Me | Me | OCH₂ | CH₂Pr^{c} |
| Cl | Cl | H | Me | Me | OCH₂ | Me |
| Cl | Cl | H | Me | H | NH | CH₂CF₃ |
| Cl | Cl | H | Me | H | NH | Me |
| Cl | Cl | H | Cl | H | OCH₂ | CHMe₂ |
| Cl | Cl | H | Cl | H | OCH₂ | CH₂Pr^{c} |
| Cl | Cl | H | Cl | H | NH | CH₂CF₃ |
| Cl | Cl | H | CN | H | NH | CH₂CF₃ |

The compounds represented by formula (I-6)

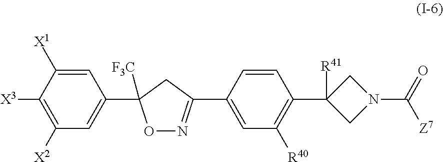

TABLE 6

| X¹ | X² | X³ | R⁴⁰ | R⁴¹ | Z⁷ |
|---|---|---|---|---|---|
| Cl | Cl | Cl | H | F | Me |
| Cl | Cl | Cl | H | F | Pr^{c} |
| Cl | Cl | Cl | H | F | NHMe |
| Cl | Cl | Cl | H | F | NHEt |
| Cl | Cl | Cl | H | F | NHPr^{c} |
| Cl | Cl | H | H | F | Pr^{c} |
| Cl | Cl | H | H | F | CHMe₂ |
| Cl | Cl | H | H | F | NHMe |
| Cl | Cl | H | H | F | NHPr^{c} |
| Cl | Cl | F | H | F | Et |
| Cl | Cl | F | H | F | Pr |
| Cl | Cl | F | H | F | CH₂Pr |
| Cl | Cl | F | H | F | CH₂CN |
| Cl | Cl | F | H | F | CH₂SMe |
| Cl | Cl | F | H | F | CH₂OMe |
| Cl | Cl | F | H | F | Bu^{c} |
| Cl | Cl | F | H | F | CHMe₂ |
| Cl | Cl | F | H | F | CH₂CF₃ |
| Cl | Cl | F | H | F | NHPr^{c} |
| Cl | Cl | F | H | F | NHMe |
| Cl | Cl | F | H | F | NHEt |
| CF₃ | Cl | Cl | H | OH | Pr^{c} |
| CF₃ | Cl | Cl | H | OH | Bu^{c} |
| CF₃ | Cl | Cl | H | F | NHMe |
| CF₃ | Cl | Cl | H | F | NHPr^{c} |
| CF₃ | Cl | Cl | H | OH | NHPr^{c} |
| CF₃ | CF₃ | H | H | OH | NHMe |
| CF₃ | CF₃ | H | H | F | NHPr^{c} |
| CF₃ | CF₃ | H | H | OH | NHPr^{c} |
| CF₃ | Cl | H | H | OH | NHEt |
| CF₃ | Cl | H | H | F | NHEt |
| Cl | Cl | F | H | F | Pr^{c} |
| Cl | Cl | F | H | F | CH₂Pr^{c} |
| Cl | Cl | H | H | F | CH₂CF₃ |
| Cl | Cl | H | H | F | CH₂OMe |
| Cl | Cl | H | H | F | Et |

The compounds represented by formula (I-7)

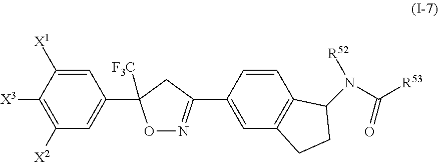

TABLE 7

| X¹ | X² | X³ | R⁵² | R⁵³ |
|---|---|---|---|---|
| Cl | Cl | H | H | Me |
| Cl | Cl | H | H | Et |
| Cl | Cl | H | H | Pr |
| Cl | Cl | H | H | CH₂CH₂OMe |
| Cl | Cl | H | H | CH₂CH₂OEt |
| Cl | Cl | H | H | CH₂CHMeOMe |
| Cl | Cl | H | H | CH₂CHMeOEt |
| Cl | Cl | H | H | CH₂CHMeOH |
| CF₃ | H | H | H | Et |
| CF₃ | H | H | H | CH₂CH₂OMe |
| CF₃ | H | H | H | CH₂CHMeOMe |
| CF₃ | H | H | H | CH₂CH₂CN |
| Cl | Cl | H | H | Pr^{c} |
| CF₃ | H | Cl | H | Et |
| CF₃ | H | Cl | H | CH₂CH₂OMe |
| CF₃ | H | H | H | CH₂CHMeOMe |
| CF₃ | H | H | H | CH₂CH₂OMe |
| Cl | Cl | H | Me | Et |

The compounds represented by formula (I-8)

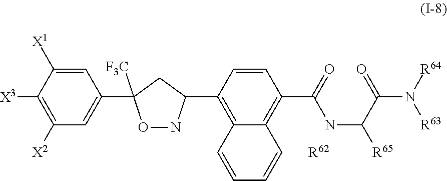

TABLE 8

| X¹ | X² | X³ | R⁶² | R⁶⁵ | R⁶⁴ | R⁶³ |
|---|---|---|---|---|---|---|
| Cl | Cl | H | H | H | H | CH₂CF₃ |
| Cl | Cl | H | H | CHMe₂ | H | CH₂CF₃ |
| Cl | Cl | H | H | H | Me | CH₂CF₃ |
| Cl | Cl | H | H | H | H | CHMe₂ |
| Cl | Cl | H | H | H | H | CH₂CHMe₂ |
| Cl | Cl | H | H | H | Me | Et |
| Cl | Cl | H | H | H | H | Et |
| Cl | Cl | H | H | H | H | CH₂CH₂Cl |
| Cl | Cl | H | H | H | H | CH₂CH₂F |
| Cl | Cl | H | H | H | H | CH₂CF₂CF₂CF₃ |
| Cl | Cl | H | H | H | H | CH₂CF₂CF₃ |
| Cl | Cl | H | H | Me | H | CH₂CF₃ |
| Cl | Cl | Cl | H | H | H | CH₂CF₃ |
| Br | Br | H | H | H | H | CH₂CF₃ |
| CF₃ | CF₃ | H | H | H | H | CH₂CF₃ |
| CF₃ | CF₃ | H | H | H | H | CHMe₂ |
| CF₃ | Br | H | H | H | H | CH₂CF₃ |
| CF₃ | Br | H | H | H | H | CHMe₂ |
| CF₃ | Cl | H | H | H | H | CH₂CF₃ |
| CF₃ | Cl | H | H | H | H | CHMe₂ |

The compounds represented by formula (I-9)

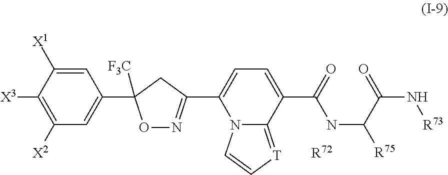

TABLE 9

| X¹ | X² | X³ | T | R⁷² | R⁷⁵ | R⁷³ |
|---|---|---|---|---|---|---|
| Cl | Cl | H | N | H | H | CH₂CF₃ |
| CF₃ | CF₃ | H | N | Me | H | CH₂CF₃ |
| CF₃ | CF₃ | H | N | H | H | CH₂CF₃ |
| CF₃ | CF₃ | H | CH | H | H | CH₂CF₃ |
| Cl | Cl | H | CH | H | H | CH₂CF₃ |
| CF₃ | Cl | H | CH | H | H | CH₂CF₃ |
| Cl | Cl | H | CMe | H | H | CH₂CF₃ |
| CF₃ | CF₃ | H | CMe | H | H | CH₂CF₃ |
| CF₃ | Cl | H | CMe | H | H | CH₂CF₃ |

The compounds represented by formula (I-10)

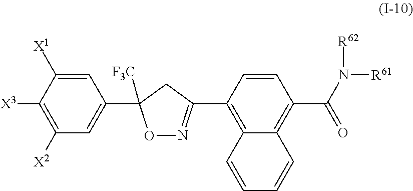

TABLE 10

| X¹ | X² | X³ | R⁶² | R⁶¹ |
|---|---|---|---|---|
| Cl | Cl | H | H | CH₂Z10 |
| Cl | Cl | H | H | CH₂CF₃ |
| Cl | Cl | H | H | CH₂CH₂SCH₃ |
| Cl | Cl | H | H | CH₂CH₂CH₂SCH₃ |
| Cl | Cl | H | H | CH₂CH₂OCH₃ |
| CF₃ | H | H | H | CH₂CH₂CH₂SCH₃ |
| CF₃ | H | H | H | CH(CH₃)CH₂OCH₃ |
| CF₃ | H | H | H | CH₂CH₂SCH₃ |

In the controlling agent of the present invention, the present isoxazoline compound may be used alone, but usually, the present isoxazoline compound is mixed with an inert carrier such as a solid carrier and a liquid carrier, and further with a surfactant or formulation auxiliaries as required, and the resulting composition is formulated into a dosage form suitable for oral administration or processed into a dosage form filled in a suitable enclosing material such as a gelatin capsule.

As a specific dosage form, the present controlling agent can be used, for example, in the forms of liquid formulations such as emulsifiable concentrate, oil formulation, oily liquid formulation, aqueous liquid formulation, solution and suspension formulation, gels, dusts, granules, paste formulation, tablets, chewable tablets, capsule formulation and syrup. The preferred dosage form is properly selected when the present controlling agent is orally administered. These formulations contain the present isoxazoline compound usually in an amount of 0.001 to 99.9% by weight.

Examples of the solid carrier which can be used in the formulation include natural or synthetic minerals such as clay, kaolin, talc, bentonite, sericite, quartz, sulfur, activated carbon, calcium carbonate, diatomaceous earth, pumice, calcite, sepiolite, dolomica, silica, alumina, vermiculite and perlite, small granules such as sawdust, corn spike, coconut shell and tobacco stem, gelatin, vaseline, methylcelluose, lanolin, lard, cacao butter, and the like.

Examples of the liquid carrier include alcohols such as methanol, ethanol, isopropyl alcohol, butanol and hexanol, polyhydric alcohols such as ethylene glycol, propylene glycol, dipropylene glycol, 1,3-butylene glycol and glycerin, ethers such as diethyl ether, ethyleneglycol dimethyl ether, diethyleneglycol monomethyl ether, diethyleneglycol monoethyl ether, propyleneglycol monomethyl ether, tetrahydrofuran and dioxane, esters such as ethyl acetate, butyl acetate and propylene carbonate, fatty acid esters such as diisopropyl adipate, diisobutyl adipate and isopropyl myristate, ketones such as acetone, methyl ethyl ketone, methyl isobutyl ketone and cyclohexanone, aromatic or aliphatic hydrocarbons such as xylene, toluene, alkylnaphthalene, phenylxylylethane, kerosene, gas oil, hexane, cyclohexane and liquid paraffin, sulfoxides such as dimethyl sulfoxide, acid amides such as N,N-dimethylformamide and N,N-dimethylacetoamide, N-methyl-2-pyrrolidone, γ-butyrolactone, vegetable oils such as soybean oil, cottonseed oil, castor oil and palm oil, plant essential oils such as orange oil, hyssop oil and lemon oil, silicone oils such as dimethyl silicone oil, highly polymerized dimethyl silicone oil, cyclic silicone oil, polyether-modified silicone oil, amino-modified silicone oil and methylphenyl silicone oil, water, and the like.

Examples of the surfactant include ampholytic surfactants, anionic surfactants, and cationic surfactants. Specific examples include the following surfactants.

Ampholytic surfactants: betaines such as laurylbetaine and stearylbetaine, imidazoline derivatives such as disodium N-lauryl-p-iminodipropionate, lecithins and the like.
Anionic surfactants: alkyl sulfates such as sodium lauryl sulfate and triethanolamine lauryl sulfate, polyoxyethylene alkyl ether sulfates such as sodium lauryl polyoxyethylene ether sulfate and triethanolamine polyoxyethylene lauryl ether sulfate, alkylbenzene sulfonates such as sodium dodecylbenzene sulfonate, polyoxyethylene alkyl ether phosphates such as sodium dipolyoxyethylene lauryl ether phosphate and sodium dipolyoxyethylene oleyl ether phosphate.
Cationic surfactants: alkyl ammonium salts such as cetyltrimethyl ammonium chloride and distearyl dimethyl ammonium chloride.

Other formulation auxiliaries include dispersing agents, antioxidants, coloring agents, light stabilizers, adhesives, and the like.

The dispersing agent includes lignin sulfonate, methylcellulose, and the like.

The antioxidant includes BHT, BHA, and the like.

The coloring agent includes food tar colors such as Red No. 2 (Amaranth), Red No. 3 (Erythrosine), Yellow No. 4 (Tartrazine), Green No. 3 (Fast Green FCF), and Blue No. 1 (Brilliant Blue FCF), iron oxide, titanium oxide, Prussian blue, alizarin dyes, azo dyes, phthalocyanine dyes, and the like.

The light stabilizer includes benzophenone compounds, benzoate compounds, benzotriazol compounds, and the like.

The adhesive includes bentonite, colloidal silicic acid, cellulose derivatives, starch derivatives, polyacrylates, natural polymers, alginic acid salts, gelatin, and the like.

The binder in the tablet and chewable tablet includes methylcellulose, carboxymethylcellulose, ethylhydroxyethylcellulose, protein derivatives such as zein and gelatin, synthetic polymers such as polyvinyl alcohol and polyvinyl pyrrolidone, starch, celluloses, and the like. In addition, fillers such as starch, microcrystalline cellulose, sugar and lactose, lubricants such as magnesium stearate and talc, and disintegrants such as starch, cellulose and carbonates may be added as necessary.

The tablet can be obtained by, for example, mixing the present isoxazoline compound, a binder and the like, and compressing the resulting mixture to a suitable size. The tablet may be coated, as necessary. Examples of the coating agent to be used for coating include coating agents containing cellulose acetate phthalate, diethyl phthalate, ethanol and dichloromethane, coating agents containing hydroxypropylcellulose, water and titanium dioxide, enteric film coating agents such as polyvinyl acetal diethylaminoacetate, food coloring agents, hydroxypropyl methylcellulose containing aqueous or non-aqueous solvents, and other film forming materials. The film coating agents may contain a plasticizer or a coloring agent.

The capsule used in the capsule formulation includes gelatin (hard or soft) capsules, hydroxypropylmethylcellulose capsules, and the like.

Also, the present controlling agent can be used in admixture or combination with commonly known other insecticide, an agent for killing animal parasitic mites, or an agent for killing endoparasites. In addition, the present controlling agent can be also used in admixture or combination with a repellent.

The amount of the present isoxazoline compound to be administered to a dog may vary depending on the type or age of the target dog or the ectoparasite to be controlled, but is usually 1 to 5000 mg/kg and preferably 1 to 100 mg/kg, per 1 kg of the living body weight of the target dog.

The number of feedings per day of dogs is generally different depending on the type, age and habit of the dog, and is usually 3 to 4 times per day for dogs being less than 6 months old, 2 to 3 times per day for dogs from 6 months old to less than 1 year old, 2 times per day for adult dogs from 1 year old to 5 years old or so, and 2 to 3 times or so per day for old dogs being 6 years old or more. In the present invention, feeding means a food intake act for the purpose of intaking nutrition, and an act of giving feed or the like for the purpose of so-called discipline or training of dogs is not included.

In the present invention, 30 minutes before the start of feeding and 120 minutes after the end of feeding are based on the act of intaking food given to a dog for the purpose of intaking nutrition. For example, when the feeding time for a dog is 20 minutes, the time prescribed in the present invention is a total of 170 minutes, from 30 minutes before the start of feeding to 120 minutes after the end of feeding, based on the dietary act. In the present invention, the case where feeding is once interrupted during feeding, the controlling agent is orally administered, and then feeding is resumed is also included. In the present invention, administration of the present isoxazoline compound to a dog is carried out separately from feeding.

Examples of the animal ectoparasites to be effectively controlled by the controlling method of the present invention include the following animal ectoparasites.

Fleas: Pulex spp. such as human flea (Pulex irritans), Ctenocephalides spp. such as cat flea (Ctenocephalides felis) and dog flea (Ctenocephalides canis), Xenopsylla spp. such as oriental rat flea (Xenopsylla cheopis), Tunga spp. such as chigoe (Tunga penetrans), Echidnophaga spp. such as chicken flea (Echidnophaga gallinacea), Nosopsyllus spp. such as European mouse flea (Nosopsyllus fasciatus), and the like. Lice: Linognathus spp. such as dog louse (Linognathus setosus). Biting lice: Trichodectes spp. such as dog biting louse (Trichodectes canis).

Hemiptera: Cimix spp. such as bedbug (Cimex lectularius) and tropical bedbug (Cimex hemipterus), Reduvius spp. such as Reduvius senilis, Rhodnius spp. such as Rhodnius prolixus, Triatoma spp. such as triatomine bug (Triatoma rubrofasciata), Panstrongylus spp., and the like.

Ticks: Amblyomma spp. such as lone star tick (Amblyomma americanum) and Ambryomma maculatum, Boophilus spp. such as cattle tick (Boophilus microplus) and Boophilus annulatus, Dermacentor spp. such as American dog tick (Dermacentor variabilis), Dermacentor taiwanicus and Dermacentor andersoni, Haemaphysalis spp. such as bush tick (Haemaphysalis longicornis), Haemaphysalis flava and Haemaphysalis campanulata, Ixodes spp. such as Ixodes ovatus, taiga tick (Ixodes persulcatus), black legged tick (Ixodes scapularis), western black-legged tick (Ixodes pacificus) and Ixodes holocyclus, Rhipicephalus spp. such as brown dog tick (Rhipicephalus sanguineus) and Rhipicephalus appendiculatus, Argas spp. such as fowl tick (Argas persicus), Ornithodorus spp. such as Ornithodorus hermsi and Ornithodorus turicata, Sarcoptes spp. such as itch mite (Sarcoptes scabiei), Otodectes spp. such as ear mite (Octodectes cynotis), Ornithonyssus spp. such as house tick (Ornithonyssus bacoti), Cheyletiella spp. such as dog cheyletid mite (Cheyletiella yasguri), Demodex spp. such as dog follicle mite (Demodex canis), Trombicula spp. such as trombiculid mite (Trombicula akamushi), Trombicula pallida and Trombicula scutellaris.

Preferred examples of the animal ectoparasites to be effectively controlled by the controlling method of the present invention include fleas, lice, or ticks.

### EXAMPLES

Hereinafter, the present invention will be described in more detail with reference to Formulation Examples of the present controlling agent and Test Examples of the present invention, and the like, but the present invention is not limited to these examples.

Next, Formulation Examples of the present controlling agent will be shown.

### Formulation Example 1

### Tablets

Two hundred mg of any one of the present isoxazoline compounds described in Tables 1 to 10, 330 mg of lactose, 118 mg of low-substituted hydroxypropyl cellulose, 142 mg of microcrystalline cellulose, 200 mg of hydrous silicon dioxide, and 10 mg of magnesium stearate were mixed together, and the resulting mixture was compression molded to obtain tablets.

### Formulation Example 2

### Tablets

One hundred mg of any one of the present isoxazoline compounds described in Tables 1 to 10, 68.75 mg of lactose, 237.5 mg of corn starch, 43.75 mg of microcrystalline cellulose, 18.75 mg of polyvinyl pyrrolidone, 28.75 mg of sodium carboxymethyl starch, and 2.5 mg of magnesium stearate are mixed together, and the resulting mixture is compressed to a suitable size to obtain tablets.

### Formulation Example 3

### Tablets

Twenty five mg of any one of the present isoxazoline compounds described in Tables 1 to 10, 73 mg of D-mannitol, 30 mg of corn starch, 7 mg of low-substituted hydroxypropyl cellulose, an appropriate amount of a 5% hydroxypropyl cellulose aqueous solution, and an appropriate amount of magnesium stearate are mixed together, and the resulting mixture is compressed to a suitable size to obtain tablets.

### Formulation Example 4

### Tablets

Four hundred mg of any one of the present isoxazoline compounds described in Tables 1 to 10, 50 mg of corn starch, 25 mg of croscarmellose sodium, 120 mg of lactose, and 5 mg of magnesium stearate are mixed together, and the resulting mixture is compressed to a suitable size to obtain tablets.

### Formulation Example 5

### Tablets

Sixty mg of any one of the present isoxazoline compounds described in Tables 1 to 10, 45 mg of microcrystalline cellulose, 4 mg of polyvinyl pyrrolidone, 4.5 mg of sodium carboxymethyl starch, 0.5 mg of magnesium stearate, and 1 mg of talc are mixed together, and the resulting mixture is compressed to a suitable size to obtain tablets.

### Formulation Example 6

### Tablets

Ten mg of any one of the present isoxazoline compounds described in Tables 1 to 10, 15 mg of starch, 127 mg of lactose, 15 mg of calcium carboxymethyl cellulose, 1 mg of magnesium stearate, and 2 mg of talc are mixed together, and the resulting mixture is compressed to a suitable size to obtain tablets.

### Formulation Example 7

### Tablets

One hundred mg of any one of the present isoxazoline compounds described in Tables 1 to 10, 600 mg of dextrin, 200 mg of potato starch, 60 mg of animal powder feed, 20 mg of sesame oil, and 20 mg of water are mixed together, and the resulting mixture is compressed to a suitable size to obtain tablets.

### Formulation Example 8

### Tablets

One hundred mg of any one of the present isoxazoline compounds described in Tables 1 to 10, 33 mg of lactose, 16 mg of corn starch, 12 mg of calcium carboxymethyl cellulose, 6 mg of methyl cellulose, and 2 mg of magnesium stearate are mixed together, and the resulting mixture is compressed to a suitable size to obtain tablets.

### Formulation Example 9

### Tablets

Ten mg of any one of the present isoxazoline compounds described in Tables 1 to 10, 46.6 mg of Fine Particles for Direct Compressing No. 209 (manufactured by Fuji Chemical Industry Co., Ltd., it consists of 20% magnesium aluminometasilicate, 30% corn starch, and 50% lactose), 24 mg of crystalline cellulose, 4 mg of calcium carboxymethyl cellulose, and 0.4 mg of magnesium stearate are mixed together, and the resulting mixture is compressed to a suitable size to obtain tablets.

### Formulation Example 10

### Tablets

Two hundred and fifty mg of any one of the present isoxazoline compounds described in Tables 1 to 10, 4.5 mg of magnesium stearate, 22.5 mg of corn starch, 9 mg of sodium starch glycolate, 4.5 mg of lauryl sodium sulfate, and 159.5 mg of microcrystalline cellulose are mixed together, and the mixture is compressed to a suitable size to obtain tablets.

### Formulation Example 11

### Tablets

Two hundred mg of any one of the present isoxazoline compounds described in Tables 1 to 10, 200 mg of lactose, 266.5 mg of potato starch, 10 mg of stearic acid, 217 mg of talc, 2.5 mg of magnesium stearate, 32 mg of colloidal silica, and an appropriate amount of ethanol are mixed together, and the mixture is compressed to a suitable size to obtain tablets.

### Formulation Example 12

### Tablets

Fifty mg of any one of the present isoxazoline compounds described in Tables 1 to 10, 7.5 mg of magnesium stearate, and 17.5 mg of microcrystalline cellulose are mixed together, and the mixture is compressed to a suitable size to obtain tablets.

### Formulation Example 13

### Tablets

Each of the tablets obtained by Formulation Examples 1 to 12 was coated with a coating agent containing a mixture solution obtained by mixing 20% acetic acid-phthalic acid cellulose, 3% diethyl phthalate, ethanol, and dichloromethane in equal amounts to obtain tablets.

### Formulation Example 14

### Capsule Formulation

Twenty five mg of any one of the present isoxazoline compounds described in Tables 1 to 10, 60 mg of lactose, 25 mg of corn starch, 6 mg of carmellose calcium, and an appropriate amount of 5% hydroxypropyl methylcellulose are mixed together, and the resulting mixture is filled into hard-shell gelatin capsules or hydroxypropyl methylcellulose capsules to obtain a capsule formulation.

### Formulation Example 15

### Capsule Formulation

Two hundred mg of any one of the present isoxazoline compounds described in Tables 1 to 10, 148 mg of lactose, and 2 mg of magnesium stearate are mixed together, and the resulting mixture is filled into hard-shell gelatin capsules or hydroxypropyl methylcellulose capsules to obtain a capsule formulation.

### Formulation Example 16

### Capsule Formulation

Two hundred and fifty mg of any one of the present isoxazoline compounds described in Tables 1 to 10, 200 mg of dry starch, and 10 mg of magnesium stearate are mixed together, and the resulting mixture is filled into hard-shell gelatin capsules or hydroxypropyl methylcellulose capsules to obtain a capsule formulation.

### Formulation Example 17

### Capsule Formulation

Two hundred and fifty mg of any one of the present isoxazoline compounds described in Tables 1 to 10, 400 mg of microcrystalline cellulose, 10 mg of fumed silicon dioxide, and 5 mg of magnesium stearate are mixed together, and the resulting mixture is filled into hard-shell gelatin capsules or hydroxypropyl methylcellulose capsules to obtain a capsule formulation.

### Formulation Example 18

### Capsule Formulation

Twenty mg of any one of the present isoxazoline compounds described in Tables 1 to 10, 251.8 mg of lactose, 2 mg of gelatin, 10 mg of corn starch, 15 mg of talc, and an appropriate amount of water are mixed together, and the resulting mixture is filled into hard-shell gelatin capsules or hydroxypropyl methylcellulose capsules to obtain a capsule formulation.

### Formulation Example 19

### Suspension Formulation for Oral Administration

To 1000 mg of any one of the present isoxazoline compounds described in Tables 1 to 10, 500 mg of fumaric acid, 2000 mg of sodium chloride, 150 mg of methylparaben, 50 mg of propylparaben, 25000 mg of granulated sugar, 13000 mg of sorbitol (70% solution), 100 mg of VeegumK (Vanderbilt Co.), 35 mg of a flavor and 500 mg of a colorant is added distillated water so as to have a final volume of 100 ml, and the ingredients are mixed to obtain a suspension formulation for oral administration.

### Formulation Example 20

### Suspension Formulation for Oral Administration

To 50 mg of any one of the present isoxazoline compounds described in Tables 1 to 10, 50 mg of sodium carboxymethylcellulose, 1.25 ml of a syrup, 0.1 ml of a benzoic acid solution, an appropriate amount of a flavor and an appropriate amount of a colorant is added water so as to have a final volume of 5 ml, and the ingredients are mixed to obtain a suspension formulation for oral administration.

### Formulation Example 21

### Liquid Formulation for Oral Administration

Five percent by weight of any one of the present isoxazoline compounds described in Tables 1 to 10 is dissolved in 5% by weight of polysorbate 85, 3% by weight of benzyl alcohol, and 30% by weight of propylene glycol. To this solution is added a phosphate buffer so as to have a pH of 6.0 to 6.5, then added water to be the final volume to obtain a liquid formulation for oral administration.

### Formulation Example 22

### Liquid Formulation for Oral Administration

Ten percent by weight of any one of the present isoxazoline compounds described in Tables 1 to 10 is homogeneously dissolved in 90% by weight of a corn oil to obtain a liquid formulation for oral administration.

### Formulation Example 23

### Liquid Formulation for Oral Administration

Five percent by weight of any one of the present isoxazoline compounds described in Tables 1 to 10 is homogeneously dissolved in 95% by weight of ethyl alcohol to obtain a liquid formulation for oral administration.

### Formulation Example 24

### Paste Formulation for Oral Administration

Five percent by weight of aluminum distearate is dispersed with heating into a mixture of 57% by weight of a fractionated palm oil and 3% by weight of polysorbate 85. This mixture is cooled to room temperature, and 25% by weight of saccharine is dispersed into the oil vehicle. To this dispersion is added 10% by weight of any one of the present isoxazoline compounds described in Tables 1 to 10 to obtain a paste formulation for oral administration.

### Formulation Example 25

### Granules for Oral Administration

Five percent by weight of any one of the present isoxazoline compounds described in Tables 1 to 10 is mixed with 95% by weight of lime stone powder, and the mixture is subjected to wet granulation to obtain granules for oral administration.

Next, it is shown that the controlling method of the present invention has an excellent controlling effect on an animal ectoparasite as test examples. The present isoxazoline compounds used in the following test examples are the following compounds.

Isoxazoline compound (1): A compound of the formula (I-1), wherein X¹ is a chlorine atom, X² is a chlorine atom, X³ is a hydrogen atom, R¹⁰ is a chlorine atom, R¹³ is a hydrogen atom, R¹² is a hydrogen atom, and R¹¹ is a cyclopropyl group.
Isoxazoline compound (2): A compound of the formula (I-3), wherein X¹ is a chlorine atom, X² is a chlorine atom, X³ is a hydrogen atom, R²⁰ is a methyl group, R²⁵ is a hydrogen atom, R²⁴ is a hydrogen atom, and R²³ is a 2,2,2-trifluoroethyl group.

### Test Example 1

### Oral Administration Test Against Dog-Infested Bush Tick (Haemaphysalis longicornis)

On the previous day before oral administration, dogs (beagle; 4 months old) were infested with 100 ticks (Haemaphysalis longicornis, young ticks). Before the oral administration, infested living ticks were counted.

Tablets of the isoxazoline compound (1) (particle diameter 23 μm) were prepared using the method of Formulation Example 1, and forcibly orally administered so that the dosage amount was 20 mg per the body weight of dog (kg) by the administration method shown in Table 11. This was referred to as a test group. On the other hand, the isoxazoline compound (1) was not administered to dogs in the non-administration group by any methods.

On the first day after administration, the number of living ticks infesting the dogs was observed. The infestation rate and the disinfestation rate were obtained using the following calculation formulae.

Infestation rate (%) on Xth day=(number of living ticks on Xth day/number of living ticks before administration)×100

Disinfestation rate (%) on Xth day=(1−infestation rate of test group on Xth day/infestation rate of non-administration group on Xth day)×100

TABLE 11

| | | Disinfestation rate (%) |
|---|---|---|
| | | of ticks on the first |
| | | day after |
| | Administration method | administration |
| Inventive plot 1 | After the dog finished eating | 100 |
| | ¼ of dog food, the tablet | |
| | was forcibly orally | |
| | administered, and the | |
| | remaining ¾ of dog food was | |
| | given. | |
| Comparative | The dog was fasted for 24 | 28 |
| plot 1 | hours, then the tablet was | |
| | forcibly orally | |
| | administered, and the dog was | |
| | fasted for further 10 hours. | |

### Test Example 2

### Oral Administration Test Against Dog-Infested Bush Tick (Haemaphysalis longicornis)

A liquid formulation of the isoxazoline compound (1) was prepared using the method of Formulation Example 23, and forcibly orally administered to dogs (beagle; 10 months old) so that the dosage amount was 5 mg per the body weight of dog (kg) by the administration method shown in Table 12. This was referred to as a test group. On the other hand, the isoxazoline compound (1) was not administered to dogs in the non-administration group by any methods.

On the seventh day after administration, dogs were infested 100 ticks. On the first day after infestation (the eighth day after administration), the number of living ticks infesting each of the dogs was observed. The infestation rate and the disinfestation rate were obtained using the above calculation formulae.

TABLE 12

| | | Disinfestation |
|---|---|---|
| | | rate (%) of |
| | | ticks on the |
| | | eighth day after |
| | Administration method | administration |
| Inventive | The dog was fasted for 24 hours, then | 100 |
| plot 3 | the liquid formulation was forcibly | |
| | orally administered, and the dog was | |
| | fed after 30 minutes of | |
| | administration. | |
| Inventive | The dog was fasted for 24 hours and then | 78 |
| plot 4 | fed (for 20 minutes), and the liquid | |
| | formulation was forcibly orally | |
| | administered after 30 minutes of the | |
| | end of feeding. | |
| Inventive | The dog was fasted for 24 hours and then | 78 |
| plot 5 | fed (for 20 minutes), and the liquid | |
| | formulation was forcibly orally | |
| | administered after 60 minutes of the | |
| | end of feeding. | |
| Inventive | The dog was fasted for 24 hours and then | 78 |
| plot 6 | fed (for 20 minutes), and the liquid | |
| | formulation was forcibly orally | |
| | administered after 120 minutes of the | |
| | end of feeding. | |
| Comparative | The dog was fasted for 24 hours, then | 59 |
| plot 3 | the liquid formulation was forcibly | |
| | orally administered, and the dog was | |
| | fed after 60 minutes of | |
| | administration. | |
| Comparative | The dog was fasted for 24 hours and then | 48 |
| plot 4 | fed (for 20 minutes), and the liquid | |
| | formulation was forcibly orally | |
| | administered after 180 minutes of the | |
| | end of feeding. | |
| Comparative | The dog was fasted for 24 hours and then | 56 |
| plot 5 | fed (for 20 minutes), and the liquid | |
| | formulation was forcibly orally | |
| | administered after 240 minutes of the | |
| | end of feeding. | |

### Test Example 3

### Oral Administration Test Against Dog-Infested Bush Tick (Haemaphysalis longicornis)

A liquid formulation of the isoxazoline compound (2) was prepared using the method of Formulation Example 23, and forcibly orally administered to dogs (beagle; 18-23 months old) so that the dosage amount was 5 mg per the body weight of dog (kg) by the administration method shown in Table 13. This was referred to as a test group. On the other hand, the isoxazoline compound (2) was not administered to dogs in the non-administration group by any methods.

On the 70th day after administration, dogs were infested 100 ticks. On the first day after infestation (the 71st day after administration), the number of living ticks infesting each of the dogs was observed. The infestation rate and the disinfestation rate were obtained using the above calculation formulae.

TABLE 13

| | | Disinfestation |
|---|---|---|
| | | rate (%) of |
| | | ticks on the |
| | | 71st day after |
| | Administration method | administration |
| Inventive | The dog was fasted for 24 hours and then | 100 |
| plot 7 | fed (for 20 minutes), and the liquid | |
| | formulation was forcibly orally | |
| | administered after 30 minutes of the | |
| | end of feeding. | |
| Inventive | The dog was fasted for 24 hours and then | 100 |
| plot 8 | fed (for 20 minutes), and the liquid | |
| | formulation was forcibly orally | |
| | administered after 120 minutes of the | |
| | end of feeding. | |
| Comparative | The dog was fasted for 24 hours and then | 56 |
| plot 6 | fed (for 20 minutes), and the liquid | |
| | formulation was forcibly orally | |
| | administered after 180 minutes of the | |
| | end of feeding. | |

The controlling method of the present invention has excellent effects as a method for controlling an ectoparasite of dogs, and thus is useful.

1. A method for controlling an ectoparasite of dogs, which comprises orally administering an ectoparasite-controlling agent comprising as an active ingredient an isoxazoline compound represented by formula (I): 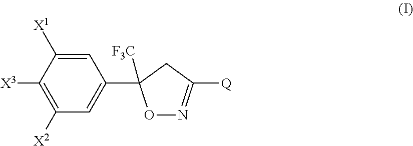
wherein X¹ represents a halogen atom or a C1-C3 haloalkyl, X² represents a hydrogen atom, a halogen atom or a C1-C3 haloalkyl, X³ represents a hydrogen atom or a halogen atom, and
Q means any group selected from Q1 to Q6 as follows: 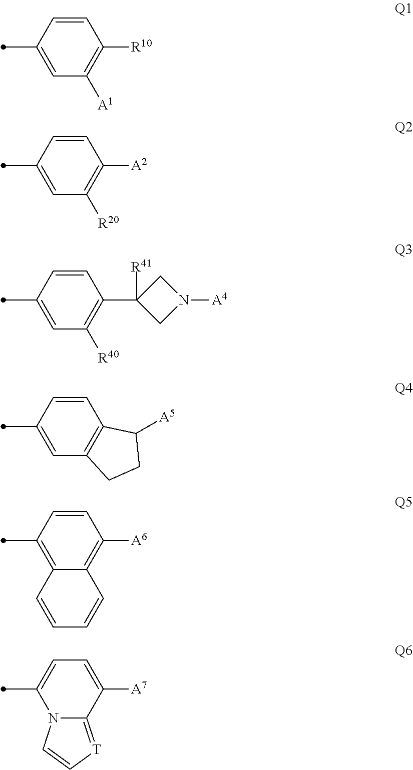
wherein
A¹ represents R¹¹—C(═O)—N(R¹²)—N(R¹³)—, R¹¹—C(═O)—N(R¹²)—CH₂— or R¹¹—C(═O)—N(R¹²)—,
R¹⁰ represents a hydrogen atom, a halogen atom or a C1-C3 alkyl,
R¹¹ represents a C1-C6 alkyl group, a C1-C6 haloalkyl group, a C3-C6 cycloalkyl group or a (C1-C6 alkoxy)C1-C6 alkyl group,
R¹² represents a hydrogen atom or a methyl group, and
R¹³ represents a hydrogen atom or a methyl group;
A² represents R²¹—N(R²²)—C(═O)—, R²³—N(R²⁴)—C(═O)—CH(R²⁵)—N(R²²)—C(═O)—, R²⁶—N(R²⁷)—N(R²²)—C(═O)—, R²⁸—N═CH—N(R²²)—C(═O)—, R²⁹—C(═O)—N(R³⁰)—CH(R³¹)—, R³²—O—N═C(R³³)—, R³⁴—NH—C(═O)—CH₂O—N═C(R³³)—, R³⁴—NH—C(═O)—NH—N═C(R³³)— or R³⁵—NH—C(═NH)—NH—N═C(R³³)—,
R²⁰ represents a hydrogen atom, a halogen atom, a nitro group, an amino group, an acetylamino group or a C1-C3 alkyl group,
R²¹ represents a C1-C6 alkyl group, a C1-C6 haloalkyl group, a (hydroxy)C1-C6 alkyl group, a (C1-C6 alkoxy)C1-C6 alkyl group, a (C1-C6 alkylthio)C1-C6 alkyl group or any one heterocyclic group selected from the following group: 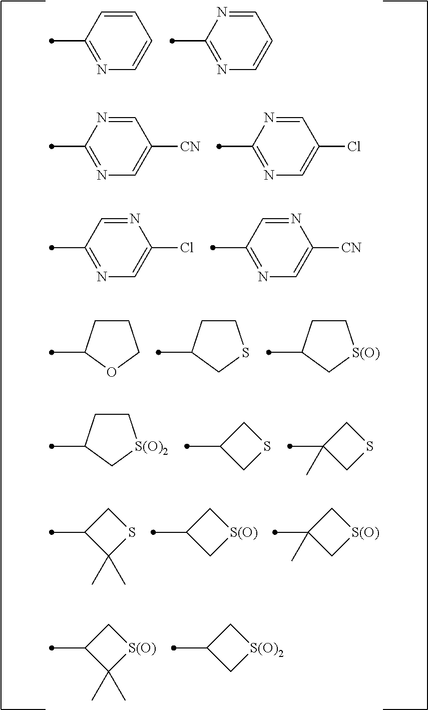
R²² represents a hydrogen atom, a (C1-C6 alkyl)carbonyl group or a (C1-C6 alkoxy)carbonyl group,
R²³ represents a C1-C6 alkyl group, a C1-C6 haloalkyl group or a (C1-C6 alkoxy)C1-C6 alkyl group,
R²⁴ represents a hydrogen atom or a C1-C3 alkyl group,
R²⁵ represents a hydrogen atom or a C1-C3 alkyl group,
R²⁶ represents a phenyl group,
R²⁷ represents a hydrogen atom or a C1-C3 alkyl group,
R²⁸ represents a C1-C3 alkoxy group,
R²⁹ represents a C1-C6 alkyl group, a C1-C6 haloalkyl group, a C3-C6 cycloalkyl group, a (C3-C6 cycloalkyl)C1-C6 alkyl group, a (C1-C6 alkoxy)C1-C6 alkyl group or a C1-C6 alkoxy group,
R³⁰ represents a hydrogen atom or a C1-C3 alkyl group,
R³¹ represents a hydrogen atom or a C1-C3 alkyl group,
R³² represents a hydrogen atom, a C1-C6 alkyl group or a C1-C6 haloalkyl group,
R³³ represents a hydrogen atom, a cyano group, a C1-C3 alkyl group, a C1-C3 alkoxy group or a (C1-C3 alkyl)carbonyl group,
R³⁴ represents a C1-C6 alkyl group, a C1-C6 haloalkyl group or a (C3-C6 cycloalkyl)C1-C6 alkyl group, and
R³⁵ represents a hydrogen atom, a C1-C6 alkyl group or a C1-C6 haloalkyl group;
A⁴ represents R⁴²—C(═O)— or R⁴²—NH—C(═O)—,
R⁴⁰ represents a hydrogen atom, a halogen atom, a nitro group, an amino group, an acetylamino group or a C1-C3 alkyl group,
R⁴¹ represents a hydrogen atom, a fluorine atom or a hydroxyl group, and
R⁴² represents a C1-C6 alkyl group, a C1-C6 haloalkyl group, a cyano(C1-C3 alkyl) group, a C3-C6 cycloalkyl group, a (C1-C6 alkoxy)C1-C6 alkyl group or a (C1-C6 alkylthio)C1-C6 alkyl group;
A⁵ represents R⁵¹—N(R⁵²)—, R⁵³—C(═O)—N(R⁵²)—, R⁵¹—N(R⁵²)—C(═O)—N(R⁵²)—, R⁵¹—O—C(═O)—N(R⁵²)— or R⁵³—C(═O)—,
R⁵¹ represents a C1-C6 alkyl group, a C1-C6 haloalkyl group or a C3-C6 cycloalkyl group,
R⁵² represents a hydrogen atom or a C1-C3 alkyl group,
R⁵³ represents a C1-C6 alkyl group, a C1-C6 haloalkyl group, a C3-C6 cycloalkyl group, a (hydroxy)C1-C6 alkyl group or a (C1-C6 alkoxy)C1-C6 alkyl group;
A⁶ represents R⁶¹—N(R⁶²)—C(═O)— or R⁶³—N(R⁶⁴)—C(═O)—CH(R⁶⁵)—N(R⁶²)—C(═O)—,
R⁶¹ represents a C1-C6 alkyl group, a C1-C6 haloalkyl group, a (hydroxy)C1-C6 alkyl group, a (C1-C6 alkoxy)C1-C6 alkyl group or a (C1-C6 alkylthio)C1-C6 alkyl group,
R⁶² represents a hydrogen atom, a (C1-C6 alkyl)carbonyl group or a (C1-C6 alkoxy)carbonyl group,
R⁶³ represents a C1-C6 alkyl group, a C1-C6 haloalkyl group or a (C1-C6 alkoxy)C1-C6 alkyl group,
R⁶⁴ represents a hydrogen atom or a C1-C6 alkyl group, and
R⁶⁵ represents a hydrogen atom or a C1-C6 alkyl group;
A⁷ represents R⁷¹—N(R⁷²)—C(═O)— or R⁷³—N(R⁷⁴)—C(═O)—CH(R⁷⁵)—N(R⁷²)—C(═O)—,
T represents a nitrogen atom or CR⁷⁶,
R⁷¹ represents a C1-C6 alkyl group, a C1-C6 haloalkyl group, a (hydroxy)C1-C6 alkyl group, a (C1-C6 alkoxy)C1-C6 alkyl group or a (C1-C6 alkylthio)C1-C6 alkyl group,
R⁷² represents a hydrogen atom, a (C1-C6 alkyl)carbonyl group or a (C1-C6 alkoxy)carbonyl group,
R⁷³ represents a C1-C6 alkyl group, a C1-C6 haloalkyl group or a (C1-C6 alkoxy)C1-C6 alkyl group,
R⁷⁴ represents a hydrogen atom or a C1-C6 alkyl group,
R⁷⁵ represents a hydrogen atom or a C1-C6 alkyl group, and
R⁷⁶ represents a hydrogen atom or a C1-C3 alkyl group,
to a dog in a period from 30 minutes immediately before the start of feeding to 120 minutes after the end of feeding.

2. The control method according to claim 1, wherein the dosage form of the ectoparasite-controlling agent is a liquid formulation, a capsule formulation, a dust, a powder, a tablet or a chewable tablet.

3. The control method according to claim 1, wherein the ectoparasite is a flea, a louse or a tick.

4. The control method according to claim 2, wherein the ectoparasite is a flea, a louse or a tick.
